(19) United States
(12) Patent Application Publication
(10) Pub. No.: US 2005/0043380 A1
(43) Pub. Date: Feb. 24, 2005

(54) Composition for treatment of malignant tumors

(76) Inventor: Richard Frederick Dechant, Sacramento, CA (US)

Correspondence Address: RICHARD FREDERICK DECHANT, 500 DUNBARTON CIRCLE, SACRAMENTO, CA, 95825, US

(21) Appl. No.: 10/642,549

(22) Filed: Aug. 18, 2003

(51) Int. Cl. A61K 31/426; A61K 31/12; A61K 31/05

(52) U.S. Cl. 514/369; 514/691; 514/734

(57) ABSTRACT

A unique composition for treatment of malignant tumors is composed of resorcinol, camphor, and sodium sulfathiazole dissolved in propylene glycol. This composition is easy to apply, and at concentrations used, causes no deleterious side effects.

### REFERENCES

Budavari ed., Merck Index, 11th edition, 1989, Monographs 1738, 8158, 8920, and 7868.

### TECHNICAL FIELD

The present invention is related to the chemical treatment of cancer. Malignant tumors susceptible to the composition are attacked via the blood stream and/or the lymph system, or by direct contact.

### BACKGROUND OF THE INVENTION

I originally formulated this composition to treat infections caused by streptococcus and staphlococcus bacteria. By chance I discovered it had properties for treating melanomas, carcinomas, and internal malignant tumors, all of which are sensitive to this composition. When the composition is applied to the skin or to sensitive mucous membranes there are no deleterious side effects other than a slight 10 to 15 second stinging sensation.

### SUMMARY OF THE INVENTION

A composition to treat malignant tumors is composed of a mixture of resorcinol, camphor, and sodium sulfathiazole dissolved in propylene glycol. When the composition is applied directly to visible cutaneous carcinomas and melanomas, it causes these diseased tissues to dry up and heal within a few days. When the composition is absorbed by contact with sublingual and rectal tissues it is dispersed in the blood and lymph systems. Upon contact with susceptible malignant tumors, it renders them inert.

### EXAMPLE 1

| COMPONENT | %WT/WT |
|---|---|
| Resorcinol | 6.7 |
| Camphor | 5.0 |
| Sodium sulfathiazole | 2.1 |
| Propylene glycol | 86.2 |

6.7 grams of resorcinol, 5.0 grams of camphor, and 2.1 grams of sodium sulfathiazole are placed in a suitable container and allowed to stand for 4 to 5 hours so that a viscous liquid is formed. 86.2 grams of propylene glycol are added and thoroughly mixed so that all of the ingredients are completely dissolved. In these proportions, resorcinol and camphor blend together easily and dissolve rapidly in propylene glycol.

The example is given solely for the purpose of illustration and is not to be construed as a limitation of the composition which may have many variations without departing from its spirit.

Method of Use;

The composition is applied to sublingual and rectal tissues by means of cotton swabs saturated with the composition. The swabs are allowed to remain in place from four to five minutes. Each swab holds about ¼ milliliter of the composition. For visible surface tumors, the composition is applied directly.

The composition differs from other anti-tumor compositions in that it requires no syntheses, no injections, and no oral ingestion. It begins action within hours and causes no apparent adverse side effects or skin irritation.

1. A method of treating carcinomas, melanomas, colorectal cancer, ovarian cancer, and all other susceptible forms of cancer, employing a composition comprising about 6.7 grams of resorcinol by weight, about 5 grams of camphor by weight, about 2.1 grams of sulfathiazole by weight, and about 86.2 grams of propylene glycol by weight;
said composition is applied employing saturated cotton swabs inserted and allowed to remain 4 to 5 minutes in contact with sublingual and rectal tissues;
for visible cutaneous tumors, the composition is applied directly.
